(19) United States
(12) Patent Application Publication
(10) Pub. No.: US 2020/0289686 A1
(43) Pub. Date: Sep. 17, 2020

(54) Using Light Fixtures For Disinfection

(71) Applicant: Signify Holding B.V., Amsterdam (NL)

(72) Inventors: Raymond George Janik, Fayetteville, GA (US); Ethan Thomas Garrett, Newnan, GA (US)

(21) Appl. No.: 16/811,283

(22) Filed: Mar. 6, 2020

Related U.S. Application Data
(60) Provisional application No. 62/816,673, filed on Mar. 11, 2019.

(51) Int. Cl. A61L 2/10; H05B 47/115; G01V 11/00

(52) U.S. Cl. CPC A61L 2/10; G01V 11/002; H05B 47/115

(57) ABSTRACT

An electrical device can include a sensor module that measures at least one parameter, where the at least one parameter is associated with determining the presence of a living being in a volume of space. The electrical device can also include a controller coupled to the sensor module, where the controller receives a measurement of the at least one parameter to determine whether the living being is in the volume of space. The electrical device can further include at least one ultraviolet (UV) light source coupled to the controller, which operates the at least one UV light source to emit UV light when the living being is not in the volume of space, and where the controller prevents the at least one UV light source from emitting the UV light when the living being is in the volume of space.

This application claims priority under 35 U.S.C. § 119 to U.S. Provisional Patent Application Ser. No. 62/816,673, titled “Using Light Fixtures For Disinfection” and filed on Mar. 11, 2019, the entire contents of which are hereby incorporated herein by reference.

### TECHNICAL FIELD

Embodiments described herein relate generally to light fixtures, and more particularly to systems, methods, and devices for using light fixtures for disinfection.

### BACKGROUND

In a number of environments (e.g., hospitals, doctor offices, certain laboratories, school classrooms, buses, urgent care clinics), bacteria, viruses, and other harmful pathogens can linger and spread, even if diligent efforts are made to disinfect those areas. An effective means of killing these bacteria, viruses, and other harmful pathogens is important for the health and safety of people who occupy these environments, but the way in which these bacteria, viruses, and other harmful pathogens are killed must also be safe for the people who occupy those environments.

### SUMMARY

In general, in one aspect, the disclosure relates to an electrical device that includes a sensor module that measures at least one parameter, where the at least one parameter is associated with determining the presence of a living being in a volume of space. The electrical device can also include a controller coupled to the sensor module, where the controller receives a measurement of the at least one parameter to determine whether the living being is in the volume of space. The electrical device can further include at least one ultraviolet (UV) light source coupled to the controller, where the controller, upon determining that the living being is not in the volume of space, operates the at least one UV light source to emit UV light into the volume of space, and where the controller, upon determining that the living being is in the volume of space, prevents the at least one UV light source from emitting the UV light into the volume of space.

In another aspect, the disclosure can generally relate to a system that includes an electrical device disposed in a volume of space. The electrical device can include a sensor module that measures at least one parameter, where the at least one parameter is associated with determining the presence of a living being in a volume of space. The electrical device can also include a controller coupled to the sensor module and the at least one UV light source, where the controller receives a measurement of the at least one parameter to determine whether the living being is in the volume of space. The electrical device can further include at least one ultraviolet (UV) light source coupled to the controller, where the controller, upon determining that the living being is not in the volume of space, operates the at least one UV light source to emit UV light into the volume of space, and where the controller, upon determining that the living being is in the volume of space, instructs the at least one UV light source to stop emitting the UV light into the volume of space.

These and other aspects, objects, features, and embodiments will be apparent from the following description and the appended claims.

### BRIEF DESCRIPTION OF THE DRAWINGS

The drawings illustrate only example embodiments of using light fixtures for disinfection and are therefore not to be considered limiting of its scope, as using light fixtures for disinfection may admit to other equally effective embodiments. The elements and features shown in the drawings are not necessarily to scale, emphasis instead being placed upon clearly illustrating the principles of the example embodiments. Additionally, certain dimensions or positions may be exaggerated to help visually convey such principles. In the drawings, reference numerals designate like or corresponding, but not necessarily identical, elements.

FIG. 1 shows a chart of the electromagnetic spectrum.

FIG. 2 shows a diagram of a lighting system that includes a light fixture in accordance with certain example embodiments.

FIG. 3 shows a computing device in accordance with certain example embodiments.

FIG. 4 shows a bottom view of a light fixture that can be used with certain example embodiments described herein.

FIGS. 5A and 5B show a graph and a table, respectively, as to the effectiveness of UV radiation exposure to E. coli bacteria using example embodiments.

FIGS. 6 through 9 show examples in accordance with example embodiments.

### DETAILED DESCRIPTION OF EXAMPLE EMBODIMENTS

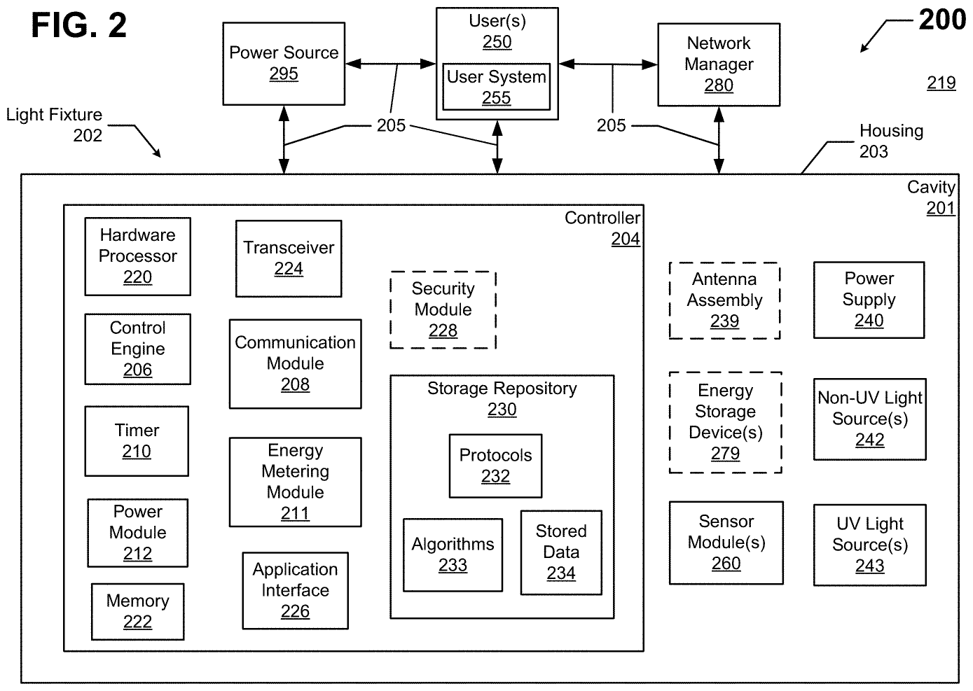

The example embodiments discussed herein are directed to systems, methods, and devices for disinfection using light fixtures. Example embodiments can be used with one or more of a number of other electrical devices in addition to, or as an alternative to, light fixtures. Such other electrical devices include one or more light sources. Examples of such other electrical devices can include, but are not limited to, a light switch, a control panel, a smoke detector, a tanning bed, a CO₂ monitor, a motion detector, a broken glass sensor, and a camera. Example embodiments can be used for a volume of space (described below with respect to FIG. 2) having any size and/or located in any environment (e.g., indoor, outdoor, hazardous, non-hazardous, high humidity, low temperature, corrosive, sterile, high vibration).

Light fixtures (or other electrical devices that use one or more light sources) described herein can use one or more of a number of different types of light sources that emit visible light that is not ultraviolet (UV), including but not limited to light-emitting diode (LED) light sources, fluorescent light sources, organic LED light sources, incandescent light sources, and halogen light sources. Such light sources are called non-UV light sources herein. Additionally, example light fixtures or other electrical devices have at least one light source capable of emitting true ultraviolet (UV) rays (e.g., 250 nm-280 nm) and/or near UV rays (e.g., 380 nm-400 nm). Such light sources are called UV light sources herein and can use any of a number of lighting technologies that are capable of emitting UV rays.

Therefore, light fixtures described herein, even in hazardous locations, should not be considered limited to a particular type of light source, both for non-UV light sources and for UV light sources. Further, example embodiments can be used in any of a number of types of light fixtures. Examples of such types of light fixtures can include, but are not limited to, a down can light, a pendant light, a street light, a Hi-Bay light, a floodlight, a beacon, a desk lamp, an under cabinet fixture, an emergency egress light, and a light integrated with a ceiling fan. Light fixtures described herein are electrical devices that can provide general illumination to a volume of space (e.g., a room, a floor, an outdoor area, a parking lot, a walkway, a stadium).

As defined herein, the term “disinfection” can have a broad meaning. Traditionally, disinfection is defined as cleaning something in order to destroy harmful microorganisms. Often, disinfection is performed using one or more chemicals. While example embodiments can be configured to disinfect in certain applications, example embodiments can also perform other similar functions, such as sanitization (defined as making something clean and hygenic) and sterilization (defined as making something free from all bacteria or other living microorganisms). As used herein, the term “disinfection” can be applied to all of these other similar functions.

In certain example embodiments, light fixtures (or other electrical devices that include light sources) are subject to meeting certain standards and/or requirements. Examples of entities that create such standards and regulations include, but are not limited to, the National Electric Code (NEC), Underwriters Laboratory (UL), the National Electrical Manufacturers Association (NEMA), the International Electrotechnical Commission (IEC), the Federal Communication Commission (FCC), the California Energy Commission, the Occupational Health and Safety Administration (OSHA), the American Safety and Health Institute (ASHI), and the Institute of Electrical and Electronics Engineers (IEEE). For example, the NEC sets standards as to electrical enclosures (e.g., light fixtures), wiring, and electrical connections. Use of example embodiments described herein meet such standards when required. In some (e.g., medical) applications, additional standards particular to that application may be met by the light fixtures or other electrical devices described herein.

If a component of a figure is described but not expressly shown or labeled in that figure, the label used for a corresponding component in another figure can be inferred to that component. Conversely, if a component in a figure is labeled but not described, the description for such component can be substantially the same as the description for the corresponding component in another figure. The numbering scheme for the various components in the figures herein is such that each component is a three-digit number and corresponding components in other figures have the identical last two digits. For any figure shown and described herein, one or more of the components may be omitted, added, repeated, and/or substituted. Accordingly, embodiments shown in a particular figure should not be considered limited to the specific arrangements of components shown in such figure.

Further, a statement that a particular embodiment (e.g., as shown in a figure herein) does not have a particular feature or component does not mean, unless expressly stated, that such embodiment is not capable of having such feature or component. For example, for purposes of present or future claims herein, a feature or component that is described as not being included in an example embodiment shown in one or more particular drawings is capable of being included in one or more claims that correspond to such one or more particular drawings herein.

Example embodiments of using light fixtures for disinfection will be described more fully hereinafter with reference to the accompanying drawings, in which example embodiments of using light fixtures for disinfection are shown. Using light fixtures for disinfection may, however, be embodied in many different forms and should not be construed as limited to the example embodiments set forth herein. Rather, these example embodiments are provided so that this disclosure will be thorough and complete, and will fully convey the scope of using light fixtures for disinfection to those of ordinary skill in the art. Like, but not necessarily the same, elements (also sometimes called components) in the various figures are denoted by like reference numerals for consistency.

Terms such as “first”, “second”, “on”, “upon”, “outer”, “inner”, “top”, “bottom”, and “within” are used merely to distinguish one component (or part of a component or state of a component) from another. Such terms are not meant to denote a preference or a particular orientation. Such terms are not meant to limit embodiments of using light fixtures for disinfection. In the following detailed description of the example embodiments, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

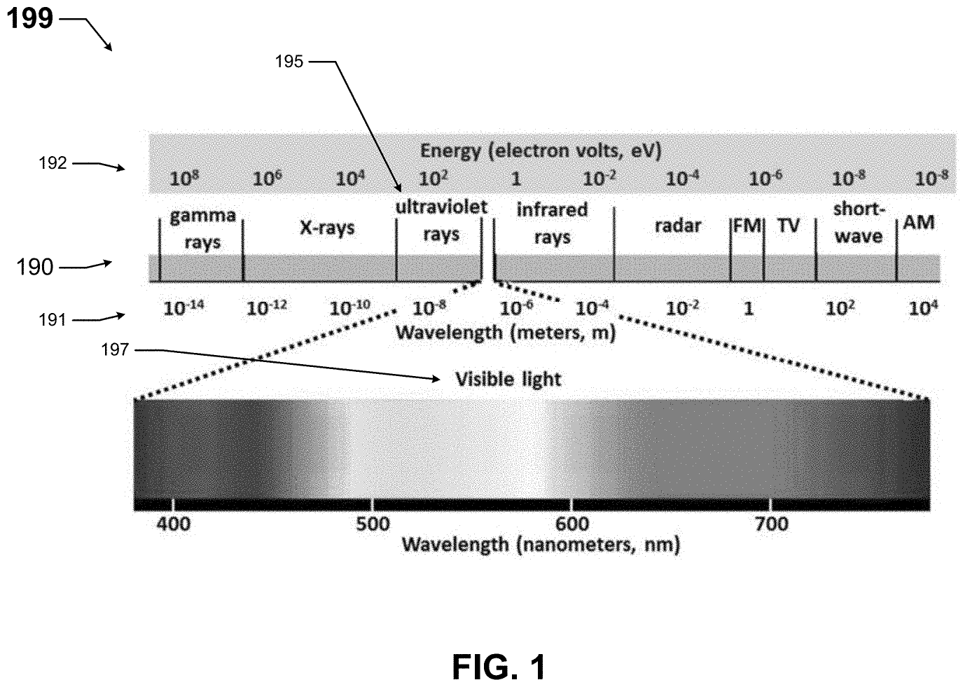

FIG. 1 shows a chart 199 of the electromagnetic spectrum 190. As discussed above, example embodiments can use both UV light sources and non-UV light sources. The chart 199 of FIG. 1 helps define the types of light in the electromagnetic spectrum 190 that can be emitted by light sources described herein. Specifically, UV rays 195 have a wavelength of between 100 nm and 400 nm. Also, UV rays 195 have a photon energy between roughly 5 eV and 100 eV. Further, as Table 1 below shows, UV rays have a frequency between approximately 750 THz and 3000 THz.

The UVA rays are also known as “near UV”. Fixtures currently in the art directed to disinfection use violet visible light and/or near UV (UVA) radiation. Exposure to UVA rays can have disinfecting results, but this usually requires an extended and continuous period of exposure for the disinfection to be effective. However, over-exposure of humans to UVA can be harmful, causing damage to DNA (for example, by the formation of free radicals and reactive oxygen species) and can cause cancer. The products currently emitting UVA radiation for disinfection largely give underwhelming results. Exposure to violet visible light is even less effective in terms of disinfection.

UVB rays can be harmful to the DNA of humans and can cause sunburn in humans. However, UVB rays are also essential for the synthesis of vitamin D in the skin of humans. General exposure of UVC rays are very harmful to human cells, but UVC rays have excellent germicidal properties. Example embodiments are designed to utilize UV light sources that emit UVC rays for disinfection.

By contrast, the rays within the visible light spectrum 197 have a wavelength between 380 nm (for violet light) and 740 nm (for red light). The photon energy for these rays in the visible light spectrum 197 is between 1.65 eV (for red light) and 3.10 eV (for violet light). Also, the wavelength of rays within the visible light spectrum is between 405 THz (for red light) and 790 THz (for violet light). These ranges are also included in Table 1 below.

TABLE 1

| Category | Subcategory | Wavelength | Frequency | Photon Energy |
|---|---|---|---|---|
| of Rays | of Rays | (nm) | (THz) | (eV) |
| UV | C | 100-280 | 1070-2997 | 4.43-12.4 |
| UV | B | 280-315 | 952-1070 | 3.94-4.43 |
| UV | A | 315-380 | 790-952 | 3.10-3.94 |
| Visible Light | Violet | 380-450 | 680-790 | 2.95-3.10 |
| Visible Light | Blue | 450-485 | 620-680 | 2.64-2.75 |
| Visible Light | Cyan | 485-500 | 600-620 | 2.48-2.52 |
| Visible Light | Green | 500-565 | 530-600 | 2.25-2.34 |
| Visible Light | Yellow | 565-590 | 510-530 | 2.10-2.17 |
| Visible Light | Orange | 590-625 | 480-510 | 2.00-2.10 |
| Visible Light | Red | 625-740 | 405-480 | 1.65-2.00 |

FIG. 2 shows a system diagram of an electrical system 200 (e.g., a lighting system) disposed in a volume of space 219, where the system 200 includes one or more sensor modules 260 (also called sensor devices 260 herein) for an electrical device 202 (e.g., a light fixture) in accordance with certain example embodiments. The electrical system 200 can include a power source 295, one or more users 250, a network manager 280, and at least one electrical device 202. In addition to the one or more sensor modules 260, the electrical device 202 can include a controller 204, one or more optional energy storage devices 279, one or more optional antenna assemblies 239 (also sometimes more simply called an antenna 239 herein), at least one power supply 240, at least one non-UV light source 242, and at least one UV light source 243.

The controller 204 can include one or more of a number of components. As shown in FIG. 2, such components can include, but are not limited to, a control engine 206, a communication module 208, a timer 210, an energy metering module 211, a power module 212, a storage repository 230, a hardware processor 220, a memory 222, a transceiver 224, an application interface 226, and, optionally, a security module 228. The components shown in FIG. 2 are not exhaustive, and in some embodiments, one or more of the components shown in FIG. 2 may not be included in an example light fixture. Any component of the example electrical device 202 can be discrete or combined with one or more other components of the electrical device 202. For example, a sensor device 260 can be remotely located from the electrical device 202, but the two can be in communication with each other.

A user 250 can be any person that interacts with electrical devices 202 (e.g., light fixtures) or components thereof (e.g., an antenna assembly 239, a sensor module 260). A user 250 can also be someone who is trying to disinfect one or more things (e.g., a room, a surface, an object). Examples of a user 250 may include, but are not limited to, a physician, a nurse, a lab technician, an engineer, an electrician, an instrumentation and controls technician, an animal (e.g., a cat, a dog, a hamster), a mechanic, an operator, a consultant, an inventory management system, an inventory manager, a foreman, a labor scheduling system, a contractor, and a manufacturer's representative.

As sometimes described herein, a user 250 can be a human or other living being. A user 250 can use one or more of a number of user systems 255 (sometimes also called user devices 255), which may include a display (e.g., a GUI). A user system 255 can be active or passive. Examples of a user system 255 can include, but are not limited to, a cell phone, a beacon, a bar code, a QR code, an identification badge, a laptop computer, an electronic tablet, and a tile. A user system 255 can broadcast communication signals using the communication links 205. In some cases, a user system 255 can also receive communication signals using the communication links 205.

A user 250 (including an associated user system 255) can interact with (e.g., send data to, receive data from) the controller 204 of the electrical device 202 via the application interface 226 (described below). A user 250 (including an associated user system 255) can also interact with the network manager 280, the power source 295, and/or one or more of the sensor modules 260. If there are multiple electrical devices 202, a user 250 (including an associated user system 255) can also interact with the controller (substantially similar to the controller 204) of those other electrical devices.

Interaction between a user 250 (including an associated user system 255) and the electrical device 202, the network manager 280, the power source 295, and the sensor modules 260 is conducted using communication links 205. Each communication link 205 can include wired (e.g., Class 1 electrical cables, Class 2 electrical cables, electrical connectors, power line carrier, DALI, RS485) and/or wireless (e.g., Wi-Fi, visible light communication, cellular networking, Bluetooth, WirelessHART, ISA100) technology. For example, a communication link 205 can be (or include) one or more electrical conductors that are coupled to the housing 203 of the electrical device 202 and to a sensor module 260. The communication link 205 can transmit signals (e.g., power signals, communication signals, control signals, data) between the electrical device 202 and a user 250 (including an associated user system 255), the network manager 280, the power source 295, and/or one or more of the sensor modules 260.

The network manager 280 is a device or component that controls all or a portion of a communication network that includes the controller 204 of the electrical device 202, additional electrical devices (e.g., light fixtures), the power source 295, and the sensor modules 260 that are communicably coupled to the controller 204. The network manager 280 can also directly or indirectly control one or more components (e.g., electrical device 202) of the system 200, or portions (e.g., UV light sources 243) thereof, using the communication network. The network manager 280 can be substantially similar to the controller 204. Alternatively, the network manager 280 can include one or more of a number of features in addition to, or altered from, the features of the controller 204 described below.

As described herein, communication with the network manager 280 can include communicating with one or more other components (e.g., another electrical device) of the system 200. In such a case, the network manager 280 can facilitate such communication. In some cases, the network manager 280 can be called by a number of other names known in the art, including but not limited to an insight manager, a master controller, and a network controller.

The power source 295 of the system 200 provides AC mains or some other form of power to the electrical device 202, as well as to one or more other components (e.g., the network manager 280, other electrical devices) of the system 200. The power source 295 can include one or more of a number of components. Examples of such components can include, but are not limited to, an electrical conductor, a coupling feature (e.g., an electrical connector), a transformer, an inductor, a resistor, a capacitor, a diode, a transistor, and a fuse. The power source 295 can be, or include, for example, a wall outlet, an energy storage device (e.g. a battery, a supercapacitor), a circuit breaker, and/or an independent source of generation (e.g., a photovoltaic solar generation system). The power source 295 can also include one or more components (e.g., a switch, a relay, a controller) that allow the power source 295 to communicate with and/or follow instructions from a user 250 (including an associated user system 255), the controller 204, one or more sensor devices 260, and/or the network manager 280.

An optional energy storage device 279 can be any of a number of rechargeable batteries or similar storage devices that are configured to charge using some source of power (e.g., the primary power provided to the electrical device 202 by the power source 295). The energy storage device 279 can use one or more of any of a number of types of storage technology, including but not limited to a battery, a flywheel, an ultracapacitor, and a supercapacitor. If the energy storage device 279 includes a battery, the battery technology can vary, including but not limited to lithium ion, nickel-cadmium, lead/acid, solid state, graphite anode, titanium dioxide, nickel cadmium, nickel metal hydride, nickel iron, alkaline, and lithium polymer.

In some cases, one or more of the energy storage devices 279 charge using a different level and/or type of power relative to the level and type of power of the primary power. In such a case, the power supply 279 can convert, invert, transform, and/or otherwise manipulate the primary power to the level and type of power used to charge the energy storage devices 279. There can be any number of energy storage devices 279.

The optional antenna assembly 239 can be any assembly of components that is used to improve the ability of the electrical device 202 (or portion thereof, such as the transceiver 224 or a sensor module 260) to send and/or receive signals with the network manager 280, the power source 295, a user 250 (including an associated user system 255), another electrical device, a remote sensor module 260, and/or some other device within the electrical system 200. The antenna assembly 239 can be used to convert electrical power into radio waves and/or convert radio waves into electrical power. An antenna assembly 239 can be used with a single component (e.g., only a sensor module 260) of the electrical device 202. Alternatively, an antenna assembly 239 can be used with multiple components (e.g., a sensor module 260, the controller 204) of the electrical device 202.

In certain example embodiments, the antenna assembly 239 includes one or more of a number of components. Such components can include, but are not limited to, a receiver, a transmitter, a switch, a balun, a block upconverter, a cable (e.g., a coaxial cable or other form of communication link 205), a counterpoise (a type of ground system), a feed, a passive radiator, a feed line, a rotator, a tuner, a low-noise block downconverter, and a twin lead. Portions of the antenna assembly 239 can be in direct communication with, or can be shared with, one or more components (e.g., the communications module 208) of the controller 204 and/or a sensor module 260. For example, the transceiver 224 of the controller 204 and/or a sensor module 260 can be in direct communication with the antenna assembly 239.

The one or more example sensor modules 260 can include one or more sensors that measure one or more parameters. Examples of types of a sensor of a sensor module 260 can include, but are not limited to, a passive infrared sensor, a photocell, a pressure sensor, an air flow monitor, a gas detector, and a resistance temperature detector. With respect to operation of a UV light source 243, examples of a parameter measured by a sensor of a sensor module 260 can include, but are not limited to, occupancy in the volume of space 219, motion in the volume of space 219, opening of a door that leads to the volume of space 219, motion in a space (e.g., a hallway) adjacent to the volume of space 219, and clearance of security access into the volume of space 219.

With respect to operation of a non-UV light source 242, examples of a parameter measured by a sensor of a sensor module 260 can include, but are not limited to, an amount of ambient light, a temperature within the housing 203 of the electrical device 202, air quality, vibration, pressure, air flow, an open door, bacteria levels, smoke (as from a fire), temperature (e.g., excessive heat, excessive cold, an ambient temperature) outside the housing 203 of the electrical device 202, detection of a gas, and humidity in the volume of space 219. In some cases, the parameter or parameters measured by a sensor of a sensor module 260 can be used to operate one or more non-UV light sources 242 and/or one or more UV light sources 243 of the electrical device 202.

A sensor device 260 can be integrated. An integrated sensor device 260 has the ability to sense and measure at least one parameter, and also the ability to directly communicate with another component (e.g., the controller 204, the network manager 280, a user system 255). The communication capability of an integrated sensor device 260 can include one or more communication devices that are configured to communicate with, for example, the controller 204 of the electrical device 202, a controller (substantially similar to the controller 204 described herein) of another electrical device, and/or the network manager 280. In some cases, an integrated sensor device 260 can be considered to be an electrical device.

Each integrated sensor device 260 can use one or more of a number of communication protocols. This allows an integrated sensor device 260 to communicate with one or more components (e.g., the controller 204, a user system 255, one or more other integrated sensor devices 260) of the system 200. The communication capability of an integrated sensor device 260 can be dedicated to the sensor device 260 and/or shared with the controller 204 of the electrical device 202. When the system 200 includes multiple integrated sensor devices 260, one integrated sensor device 260 can communicate, directly or indirectly, with one or more of the other integrated sensor devices 260 in the system 200.

If the communication capability of an integrated sensor device 260 is dedicated to the sensor device 260, then the integrated sensor device 260 can include one or more components (e.g., a transceiver 224, a communication module 208, antenna assembly 239), or portions thereof, that are substantially similar to the corresponding components described above with respect to the controller 204 or other portions of the electrical device 202. A sensor device 260, whether integrated or not, can be associated with the electrical device 202 and/or another electrical device in the system 200. A sensor device 260 can be located within the housing 203 of the electrical device 202, disposed on the housing 203 of the electrical device 202, or located outside the housing 203 of the electrical device 202.

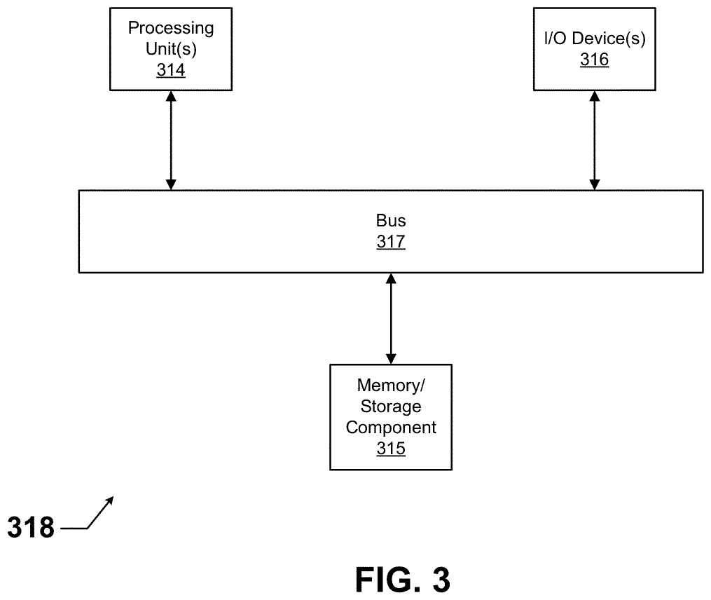

In certain example embodiments, a sensor module 260 can include an energy storage device (e.g., a battery) that is used to provide power, at least in part, to some or all of the sensor module 260. In such a case, the energy storage device can be the same as, or independent of, the energy storage device 279, described above, of the electrical device 202. The energy storage device of the sensor module 260 can operate at all times or only when a primary source of power to the electrical device 202 is interrupted. In some cases, a sensor module 260 can utilize or include one or more components (e.g., memory 222, storage repository 230, transceiver 224) found in the controller 204. In such a case, the controller 204 can provide the functionality of these components used by the sensor module 260. Alternatively, as with an intergrated sensor module 260, a sensor module 260 can include, either on its own or in shared responsibility with the controller 204, one or more of the components of the controller 204. In such a case, the sensor module 260 can correspond to a computer system as described below with regard to FIG. 3.

A sensor module 260 in example embodiments can be at least partially disposed within the housing 203 of the electrical device 202. As another example, an entire sensor module 260 (or portions thereof) can be disposed on (integrated with) the housing 203 of the electrical device 202. Example sensor modules 260 (or portions thereof) described herein can be printed on an outer surface of the housing 203 of the electrical device 202 or printed on an information medium (e.g., a warning label, a nameplate) that is adhered or otherwise coupled to the outer surface of the housing 203 of the electrical device 202.

A user 250 (including an associated user system 255), the network manager 280, the power source 295, one or more other electrical devices, and/or the sensor modules 260 can interact with the controller 204 of the electrical device 202 using the application interface 226 in accordance with one or more example embodiments. Specifically, the application interface 226 of the controller 204 receives data (e.g., information, communications, instructions, updates to firmware) from and sends data (e.g., information, communications, instructions) to a user 250 (including an associated user system 255), one or more other electrical devices, the network manager 280, the power source 295, and/or each sensor module 260.

A user 250 (including an associated user system 255), one or more other electrical devices, the network manager 280, the power source 295, and/or each sensor module 260 can include an interface to receive data from and send data to the controller 204 in certain example embodiments. Examples of such an interface can include, but are not limited to, a graphical user interface, a touchscreen, an application programming interface, a keyboard, a monitor, a mouse, a web service, a data protocol adapter, some other hardware and/or software, or any suitable combination thereof.

The controller 204, a user 250 (including an associated user system 255), one or more other electrical devices, the network manager 280, the power source 295, and/or the sensor modules 260 can use their own system or share a system in certain example embodiments. Such a system can be, or contain a form of, an Internet-based or an intranet-based computer system that is capable of communicating with various software. A computer system includes any type of computing device and/or communication device, including but not limited to the controller 204. Examples of such a system can include, but are not limited to, a desktop computer with a Local Area Network (LAN), a Wide Area Network (WAN), Internet or intranet access, a laptop computer with LAN, WAN, Internet or intranet access, a smart phone, a server, a server farm, an android device (or equivalent), a tablet, smartphones, and a personal digital assistant (PDA). Such a system can correspond to a computer system as described below with regard to FIG. 3.

Further, as discussed above, such a system can have corresponding software (e.g., user software, sensor device software, controller software, network manager software). The software can execute on the same or a separate device (e.g., a server, mainframe, desktop personal computer (PC), laptop, PDA, television, cable box, satellite box, kiosk, telephone, mobile phone, or other computing devices) and can be coupled by the communication network (e.g., Internet, Intranet, Extranet, LAN, WAN, or other network communication methods) and/or communication channels, with wire and/or wireless segments according to some example embodiments. The software of one system can be a part of, or operate separately but in conjunction with, the software of another system within the system 200.

The electrical device 202 can include a housing 203. The housing 203 can include at least one wall that forms a cavity 201. In some cases, the housing 203 can be designed to comply with any applicable standards so that the electrical device 202 can be located in a particular environment (e.g., outdoors, in an indoor “clean room”). The housing 203 of the electrical device 202 can be used to house one or more components of the electrical device 202, including one or more components of the controller 204. For example, as shown in FIG. 2, the controller 204 (which in this case includes the control engine 206, the communication module 208, the timer 210, the energy metering module 211, the power module 212, the storage repository 230, the hardware processor 220, the memory 222, the transceiver 224, the application interface 226, and the optional security module 228), one or more of the sensor modules 260, one or more optional antenna assemblies 239, the power supply 240, the non-UV light sources 242, and the UV light sources 243 are disposed, at least in part, in the cavity 201 formed by the housing 203.

In alternative embodiments, any one or more of these or other components of the electrical device 202 can be disposed on the housing 203 and/or located remotely from the housing 203. For instance, an example sensor module 260 (or portion thereof) can be integrated with the housing 203. As another example, the UV light sources 243 can be part of a separate electrical device from electrical device 202, where the operation of the UV light sources 243 is controlled by the controller 204 and one or more of the sensors 260.

The storage repository 230 can be a persistent storage device (or set of devices) that stores software and data used to assist the controller 204 in communicating with a user 250 (including an associated user system 255), one or more other electrical devices, the network manager 280, the power source 295, and one or more sensor modules 260 within the system 200. In one or more example embodiments, the storage repository 230 stores one or more protocols 232, one or more algorithms 233, and stored data 234. The protocols 232 can be any procedures (e.g., a series of method steps), logic steps, and/or other similar operational procedures that the control engine 206 of the controller 204 follows based on certain conditions at a point in time.

The protocols 232 can also include any of a number of communication protocols 232 that are used to send and/or receive data between the controller 204 and a user 250 (including an associated user system 255), one or more other electrical devices, the network manager 280, the power source 295, and one or more sensor modules 260. One or more of the protocols 232 used for communication can be a time-synchronized protocol. Examples of such time-synchronized protocols can include, but are not limited to, a highway addressable remote transducer (HART) protocol, a wirelessHART protocol, and an International Society of Automation (ISA) 100 protocol.

In this way, one or more of the protocols 232 used for communication can provide a layer of security to the data transferred within the system 200. Other protocols 232 used for communication can be associated with the use of Wi-Fi, Zigbee, visible light communication, cellular networking, ultra-wideband, Bluetooth Low Energy (BLE), and Bluetooth. One or more protocols 232 can facilitate communication between a sensor module 260 and the control engine 206 of the controller 204.

The algorithms 233 can be any formulas, mathematical models, forecasts, simulations, and/or other similar computational instruments that the control engine 206 of the controller 204 utilizes based on certain conditions at a point in time. One or more algorithms 233 can be used in conjunction with, or as a result of following, one or more protocols 231. An example of an algorithm 233 is determining the effectiveness of UV light emitted by the UV light sources 243 in killing harmful bacteria. Another example of an algorithm 233 is estimating the minimal amount of time that the UV light sources 243 should operate to effectively kill harmful bacteria.

Algorithms 233 can be focused on certain components of the electrical device 202. For example, one or more algorithms 233 can use parameters measured by one or more sensor modules 260. As a specific example, a protocol 231 can be used by the control engine 206 to instruct a sensor module 260 (in some cases, using an antenna assembly 239) to measure a parameter (e.g., occupancy of a room, opening of a door to a room), for the sensor module 260 to send the measurement to the control engine 206, for the control engine 206 to analyze the measurement using one or more algorithms 233, and for the control engine 206 to take an action (e.g., instruct, using a protocol 232, one or more of the UV light sources 243 to stop operating) based on the result (stored as stored data 234) of the algorithm 233.

Stored data 234 can be any data associated with the electrical device 202 (including other light fixtures and/or any components thereof), any measurements taken by the sensor modules 260, measurements taken by the energy metering module 211, threshold values, user preferences and settings, results of previously run or calculated algorithms 232, and/or any other suitable data. Such data can be any type of data, including but not limited to historical data (e.g., historical data for the electrical device 202, historical data for other electrical devices), present data (e.g., calculations, measurements taken by the energy metering module 211, and measurements taken by one or more sensor modules 260), and forecast data. The stored data 234 can be associated with some measurement of time derived, for example, from the timer 210.

Examples of a storage repository 230 can include, but are not limited to, a database (or a number of databases), a file system, a hard drive, flash memory, cloud-based storage, some other form of solid state data storage, or any suitable combination thereof. The storage repository 230 can be located on multiple physical machines, each storing all or a portion of the protocols 232, the algorithms 233, and/or the stored data 234 according to some example embodiments. Each storage unit or device can be physically located in the same or in a different geographic location.

The storage repository 230 can be operatively connected to the control engine 206. In one or more example embodiments, the control engine 206 includes functionality to communicate with a user 250 (including an associated user system 255), one or more other electrical devices, the network manager 280, the power source 295, and the sensor modules 260 in the system 200. More specifically, the control engine 206 sends information to and/or receives information from the storage repository 230 in order to communicate with a user 250 (including an associated user system 255), one or more other electrical devices, the network manager 280, the power source 295, and the sensor modules 260. As discussed below, the storage repository 230 can also be operatively connected to the communication module 208 in certain example embodiments.

In certain example embodiments, the control engine 206 of the controller 204 controls the operation of one or more components (e.g., the communication module 208, the timer 210, the transceiver 224) of the controller 204. For example, the control engine 206 can activate the communication module 208 when the communication module 208 is in “sleep” mode and when the communication module 208 is needed to send data received from another component (e.g., a sensor module 260, a user system 255 of a user 250) in the system 200.

As another example, the control engine 206 can acquire the current time using the timer 210. The timer 210 can enable the controller 204 to control the electrical device 202 even when the controller 204 has no communication with the network manager 280. As yet another example, the control engine 206 can determine, based on measurements made by one or more sensor modules 260, when it is safe (e.g., no people in the area) to operate the one or more UV light sources 243, and then operate those UV light sources 243 for as long as the safe condition exists. Similarly, the control engine 206 can determine, based on measurements made by one or more sensor modules 260, when it is no longer safe (e.g., people are approaching the area) or necessary (e.g., a sufficient amount of time (measured by the timer 210) has elapsed) to operate the one or more UV light sources 243, and then cease operation of those UV light sources 243.

The control engine 206 of the controller 204 can operate the non-UV light sources 242 in a special way when the UV light sources 243 are simultaneously being operated. Since UV light emitted by the UV light sources 243 is not visible to the human eye, a human user 250 entering the volume of space 219 would not know whether the UV light sources 243 are operating. As an indication to a user 250 that the UV light sources 243 are operating, the controller can instruct the non-UV light sources 242 to emit a non-standard color (e.g., red, blue) into the volume of space 219.

The control engine 206 of the controller 204 can communicate, in some cases using the antenna assembly 239, with one or more of the example sensor modules 260 and make determinations based on measurements made by the example sensor modules 260. For example, the control engine 206 can use one or more protocols 232 and/or algorithms 233 to facilitate communication with a sensor module 260. As a specific example, the control engine 206 can use one or more protocols 232 to instruct a sensor module 260 to measure a parameter, for the sensor module 260 to send the measurement to the control engine 206, for the control engine 206 to analyze, using one or more algorithms 233, the measurement, (stored as stored data 234) and for the control engine 206 to take an action (e.g., instruct, using a protocol 232, one or more other components (e.g., the UV light sources 243) of the electrical device 202 to operate) based on the result (stored as stored data 234) of the analysis.

The control engine 206 can also send and/or receive communications. As a specific example, the control engine 206 can use one or more algorithms 233 to receive (using a protocol 232) a signal (e.g., received by the antenna assembly 239), for the control engine 206 to analyze the signal, and for the control engine 206 to take an action (e.g., instruct one or more other components of the electrical device 202 to operate) based on the result of the analysis. As another specific example, the control engine 206 can use one or more protocols 232 and/or algorithms 233 to determine that a communication to a device external to the electrical device 202 needs to be sent, and to send a communication signal (using a protocol 232 and saved as stored data 234), in some cases using the antenna assembly 239.

As discussed above, the control engine 206 can in some cases control one or more additional electrical devices in conjunction with controlling the UV light sources 243 of the electrical device 202. For example, after the control engine 206 determines, based on measurements made by a sensor module 260, that a room is empty of human beings, and before the control engine 206 turns on the UV light sources 243 of the electrical device 202, the control engine 206 can use the communication links 205 to operate one or more electronic door locks for all doors providing access to the volume of space 219 so that entry through the corresponding doors is prohibited for as long as the UV light sources 243 are emitting UV light into the volume of space 219.

Similarly, when the control engine 206 has determined (e.g., based on an amount of time that the UV light sources 243 are emitting UV light into the volume of space 219, based on authorization of a user 250 to enter the volume of space 219) that the UV light sources 243 need to be turned off, the control engine 206 can turn off the UV light sources 243 and then subsequently operate one or more of the electronic door locks so that a user 250 can safely access the volume of space 219. In other words, the control engine 206 can implement any of a number of safety protocols, using one or more other electrical devices, to ensure that a user 250 is not exposed to UV light emitted by the UV light sources 243.

The control engine 206 can provide control, communication, and/or other similar signals to a user 250 (including an associated user system 255), one or more other electrical devices, the network manager 280, the power source 295, and one or more of the sensor modules 260. Similarly, the control engine 206 can receive control, communication, and/or other similar signals from a user 250 (including an associated user system 255), one or more other electrical devices, the network manager 280, the power source 295, and one or more of the sensor modules 260. The control engine 206 can control each sensor module 260 automatically (for example, based on one or more algorithms stored in the control engine 206) and/or based on control, communication, and/or other similar signals received from another device through a communication link 205. The control engine 206 may include a printed circuit board, upon which the hardware processor 220 and/or one or more discrete components of the controller 204 are positioned.

In certain embodiments, the control engine 206 of the controller 204 can communicate with one or more components of a system external to the system 200. For example, the control engine 206 can interact with an inventory management system by ordering an electrical device 202 (or one or more components thereof) to replace the electrical device 202 (or one or more components thereof) that the control engine 206 has determined to fail or be failing. As another example, the control engine 206 can interact with a workforce scheduling system by scheduling a maintenance crew to repair or replace the electrical device 202 (or portion thereof) when the control engine 206 determines that the electrical device 202 or portion thereof requires maintenance or replacement. In this way, the controller 204 is capable of performing a number of functions beyond what could reasonably be considered a routine task.

In certain example embodiments, the control engine 206 can include an interface that enables the control engine 206 to communicate with one or more components (e.g., power supply 240) of the electrical device 202. For example, if the power supply 240 of the electrical device 202 operates under IEC Standard 62386, then the power supply 240 can have a serial communication interface that will transfer data (e.g., stored data 234) measured by the sensor modules 260. In such a case, the control engine 206 can also include a serial interface to enable communication with the power supply 240 within the electrical device 202. Such an interface can operate in conjunction with, or independently of, the protocols 232 used to communicate between the controller 204 and a user 250 (including an associated user system 255), one or more other electrical devices, the network manager 280, the power source 295, and the sensor modules 260.

The control engine 206 (or other components of the controller 204) can also include one or more hardware components and/or software elements to perform its functions. Such components can include, but are not limited to, a universal asynchronous receiver/transmitter (UART), a serial peripheral interface (SPI), a direct-attached capacity (DAC) storage device, an analog-to-digital converter, an inter-integrated circuit (I2C), and a pulse width modulator (PWM).

The communication module 208 of the controller 204 determines and implements the communication protocol (e.g., from the protocols 232 of the storage repository 230) that is used when the control engine 206 communicates with (e.g., sends signals to, receives signals from) a user 250 (including an associated user system 255), one or more other electrical devices, the network manager 280, the power source 295, and/or one or more of the sensor modules 260. In some cases, the communication module 208 accesses the stored data 234 to determine which protocol 232 is used to communicate with the sensor module 260 associated with the stored data 234. In addition, the communication module 208 can interpret the communication protocol of a communication received by the controller 204 so that the control engine 206 can interpret the communication.

The communication module 208 can send and receive data between the network manager 280, the power source 295, one or more other electrical devices, the sensor modules 260, and/or the users 250 (including an associated user system 255) and the controller 204. The communication module 208 can send and/or receive data in a given format that follows a particular protocol 232. The control engine 206 can interpret the data packet received from the communication module 208 using the protocol 232 information stored in the storage repository 230. The control engine 206 can also facilitate the data transfer between one or more sensor modules 260 and the network manager 280 or a user 250 (including an associated user system 255) by converting the data into a format understood by the communication module 208.

The communication module 208 can send data (e.g., protocols 232, algorithms 233, stored data 234, operational information, alarms) directly to and/or retrieve data directly from the storage repository 230. Alternatively, the control engine 206 can facilitate the transfer of data between the communication module 208 and the storage repository 230. The communication module 208 can also provide encryption to data that is sent by the controller 204 and decryption to data that is received by the controller 204. The communication module 208 can also provide one or more of a number of other services with respect to data sent from and received by the controller 204. Such services can include, but are not limited to, data packet routing information and procedures to follow in the event of data interruption.

The timer 210 of the controller 204 can track clock time, intervals of time, an amount of time, and/or any other measure of time. The timer 210 can also count the number of occurrences of an event, whether with or without respect to time. Alternatively, the control engine 206 can perform the counting function. The timer 210 is able to track multiple time measurements concurrently. The timer 210 can track time periods based on an instruction received from the control engine 206, based on an instruction received from the user 250, based on an instruction programmed in the software for the controller 204, based on some other condition or from some other component, or from any combination thereof.

The timer 210 can be configured to track time when there is no power delivered to the controller 204 (e.g., the power module 212 malfunctions) using, for example, a super capacitor or a battery backup. In such a case, when there is a resumption of power delivery to the controller 204, the timer 210 can communicate any aspect of time to the controller 204. In such a case, the timer 210 can include one or more of a number of components (e.g., a super capacitor, an integrated circuit) to perform these functions.

The energy metering module 211 of the controller 204 measures one or more components of power (e.g., current, voltage, resistance, VARs, watts) at one or more points within the electrical device 202. The energy metering module 211 can include any of a number of measuring devices and related components, including but not limited to a voltmeter, an ammeter, a power meter, an ohmmeter, a current transformer, a potential transformer, and electrical wiring. The energy metering module 211 can measure a component of power continuously, periodically, based on the occurrence of an event, based on a command received from the control module 206, and/or based on some other factor. For purposes herein, the energy metering module 211 can be considered a type of sensor (e.g., sensor module 260). In this way, a component of power measured by the energy metering module 211 can be considered a parameter herein.

In certain example embodiments, the power module 212 of the controller 204 receives power from the power supply 240 and manipulates (e.g., transforms, rectifies, inverts) that power to provide the manipulated power to one or more other components (e.g., timer 210, control engine 206) of the controller 204. Alternatively, in certain example embodiments, the power module 212 can provide power to the power supply 240 and/or other components (e.g., an antenna assembly 239, an energy storage device 279) of the electrical device 202. The power module 212 can include one or more of a number of single or multiple discrete components (e.g., transistor, diode, resistor), and/or a microprocessor. The power module 212 may include a printed circuit board, upon which the microprocessor and/or one or more discrete components are positioned. In some cases, the power module 212 can include one or more components that allow the power module 212 to measure one or more elements of power (e.g., voltage, current) that is delivered to and/or sent from the power module 212.

The power module 212 can include one or more components (e.g., a transformer, a diode bridge, an inverter, a converter) that receives power (for example, through an electrical cable) from the power supply 240 (or in some cases from the power source 295) and generates power of a type (e.g., alternating current, direct current) and level (e.g., 12V, 24V, 120V) that can be used by the other components of the controller 204 and/or one or more other components of the electrical device 202. The power module 212 can use a closed control loop to maintain a preconfigured voltage or current with a tight tolerance at the output. The power module 212 can also protect the rest of the electronics (e.g., hardware processor 220, transceiver 224) in the electrical device 202 from surges generated in the line.

In addition, or in the alternative, the power module 212 can be or include a source of power in itself to provide signals to the other components of the controller 204 and/or the power supply 240. For example, the power module 212 can be or include a battery. As another example, the power module 212 can be or include a localized photovoltaic power system. The power module 212 can also have sufficient isolation in the associated components of the power module 212 (e.g., transformers, opto-couplers, current and voltage limiting devices) so that the power module 212 is certified to provide power to an intrinsically safe circuit.

In certain example embodiments, the power module 212 of the controller 204 can also provide power and/or control signals, directly or indirectly, to one or more of the sensor modules 260. In such a case, the control engine 206 can direct the power generated by the power module 212 to the sensor modules 260 of the electrical device 202. In this way, power can be conserved by sending power to the sensor modules 260 of the electrical device 202 when those devices need power, as determined by the control engine 206.

The hardware processor 220 of the controller 204 executes software, algorithms, and firmware in accordance with one or more example embodiments. Specifically, the hardware processor 220 can execute software on the control engine 206 or any other portion of the controller 204, as well as software used by a user 250 (including an associated user system 255), one or more other electrical devices, the network manager 280, the power source 295, and/or one or more of the sensor modules 260. The hardware processor 220 can be or include an integrated circuit, a central processing unit, a multi-core processing chip, SoC, a multi-chip module including multiple multi-core processing chips, or other hardware processor in one or more example embodiments. The hardware processor 220 is known by other names, including but not limited to a computer processor, a microprocessor, and a multi-core processor.

In one or more example embodiments, the hardware processor 220 executes software instructions stored in memory 222. The memory 222 includes one or more cache memories, main memory, and/or any other suitable type of memory. The memory 222 can include volatile and/or non-volatile memory. The memory 222 is discretely located within the controller 204 relative to the hardware processor 220 according to some example embodiments. In certain configurations, the memory 222 can be integrated with the hardware processor 220.

In certain example embodiments, the controller 204 does not include a hardware processor 220. In such a case, the controller 204 can include, as an example, one or more field programmable gate arrays (FPGA), one or more insulated-gate bipolar transistors (IGBTs), one or more complex programmable logic devices (CPLDs), programmable array logics (PALs), one or more digital signal processors (DSPs), and one or more integrated circuits (ICs). Using FPGAs, IGBTs, CPLDs, PALs, DSPs, ICs, and/or other similar devices known in the art allows the controller 204 (or portions thereof) to be programmable and function according to certain logic rules and thresholds without the use of a hardware processor. Alternatively, FPGAs, IGBTs, CPLDs, PALs, DSPs, ICs, and/or similar devices can be used in conjunction with one or more hardware processors 220.

The transceiver 224 of the controller 204 can send and/or receive control and/or communication signals. Specifically, the transceiver 224 can be used to transfer data between the controller 204 and a user 250 (including an associated user system 255), one or more other electrical devices, the network manager 280, the power source 295, and/or the sensor modules 260. The transceiver 224 can use wired and/or wireless technology. The transceiver 224 can be configured in such a way that the control and/or communication signals sent and/or received by the transceiver 224 can be received and/or sent by another transceiver that is part of a user 250 (including an associated user system 255), one or more other electrical devices, the network manager 280, the power source 295, and/or the sensor modules 260. The transceiver 224 can use any of a number of signal types, including but not limited to radio signals. In some cases, the transceiver 224 can be part of, or at least in communication with, the antenna assembly 239.

When the transceiver 224 uses wireless technology, any type of wireless technology can be used by the transceiver 224 in sending and receiving signals. Such wireless technology can include, but is not limited to, Wi-Fi, Zigbee, visible light communication, cellular networking, ultra-wideband, BLE, and Bluetooth. The transceiver 224 can use one or more of any number of suitable communication protocols (e.g., ISA100, HART) when sending and/or receiving signals. Such communication protocols can be stored in the protocols 232 of the storage repository 230. Further, any transceiver information for a user 250 (including an associated user system 255), one or more other electrical devices, the network manager 280, the power source 295, and/or the sensor modules 260 can be part of the stored data 234 (or similar areas) of the storage repository 230.

Optionally, in one or more example embodiments, the security module 228 secures interactions between the controller 204, a user 250 (including an associated user system 255), one or more other electrical devices, the network manager 280, the power source 295, and/or the sensor modules 260. More specifically, the security module 228 authenticates communication from software based on security keys verifying the identity of the source of the communication. For example, user software may be associated with a security key enabling the software of a user system 255 of a user 250 to interact with the controller 204 and/or the sensor modules 260. Further, the security module 228 can restrict receipt of information, requests for information, and/or access to information in some example embodiments.

As mentioned above, aside from the controller 204 and its components, the electrical device 202 can include a power supply 240, one or more non-UV light sources 242, and one or more UV light sources 243. Alternatively, one electrical device 202 can include non-UV light sources 242, while another electrical device 202 in communication with the first electrical device 202 can include UV light sources 243. The non-UV light sources 242 of the electrical device 202 are devices and/or components typically found in a light fixture or other electrical device 202 to allow the electrical device 202 to operate.

The electrical device 202 can have one or more of any number and/or type of non-UV light sources 242 and/or UV light sources 243. The non-UV light sources 242 and the UV light sources 243 can include any of a number of components, including but not limited to a local control module, a light source, a light engine, a heat sink, an electrical conductor or electrical cable, a terminal block, a lens, a diffuser, a reflector, an air moving device, a baffle, a dimmer, and a circuit board. In some cases, the electrical device 202 can have no non-UV light sources 242 and only UV light sources 243.

A non-UV light source 242 can use one or more of any type of lighting technology, including but not limited to LED, incandescent, sodium vapor, and fluorescent. A UV light source 243 can use one or more of any type of lighting technology, including but not limited to lights used in tanning booths, black lights, curing lamps, germicidal lamps, mercury vapor lamps, halogen lights, high-intensity discharge lamps, specialized LEDs, arc lamps, fluorescent and incandescent sources, and some types of lasers. The UV radiation emitted by a UV light source 243 can include UVA radiation, UVB radiation, and/or UVC radiation.

In some cases, the lighting technology used for a UV light source 243 is the same as the lighting technology used for a non-UV light source 242. In such a case, the UV light source 243 and the non-UV light source 242 can be part of the same full-spectrum chip (or similar device or technology). The control engine 206 of the controller 204 can then control (or include) the full-spectrum chip to have the same light source alternate between emitting UV light and non-UV light.

The power supply 240 of the electrical device 202 provides power to the controller 204, one or more of the optional antenna assemblies 239, one or more of the optional energy storage devices 279, one or more of the sensor modules 260, one or more of the non-UV light sources 242, and/or one or more of the UV light sources 243. The power supply 240 can be called by any of a number of other names, including but not limited to a driver, a LED driver, and a ballast. The power supply 240 can be substantially the same as, or different than, the power module 212 of the controller 204. For example, the power supply 240 can include one or more of a number of single or multiple discrete components (e.g., transistor, diode, resistor), and/or a microprocessor. As another example, the power supply 240 may include a printed circuit board, upon which the microprocessor and/or one or more discrete components are positioned, and/or a dimmer.

The power supply 240 can include one or more components (e.g., a transformer, a diode bridge, an inverter, a converter) that receives power (for example, through an electrical cable) from the power source 295 (or another source external to the electrical device 202) and generates power of a type (e.g., alternating current, direct current) and level (e.g., 12V, 24V, 120V) that can be used by the controller 204, the optional antenna assemblies 239, the optional energy storage devices 279, the sensor modules 260, the non-UV light sources 242, and/or the UV light sources 243. In addition, or in the alternative, the power supply 240 can receive power from the power module 212 of the controller 204. In addition, or in the alternative, the power supply 240 can be or include a source of power in itself. For example, the power supply 240 can be or include a battery, a localized photovoltaic power system, or some other source of independent power.

As stated above, the electrical device 202 can be placed in any of a number of environments. In such a case, the housing 203 of the electrical device 202 can be configured to comply with applicable standards for any of a number of environments. This compliance with applicable standards can be upheld when at least a portion of an example sensor module 260 is integrated with the housing 203 of the electrical device 202.

FIG. 3 illustrates one embodiment of a computing device 318 that implements one or more of the various techniques described herein, and which is representative, in whole or in part, of the elements described herein pursuant to certain exemplary embodiments. For example, the controller 204 of FIG. 2 and its various components (e.g., hardware processor 220, memory 222, control engine 206) can be considered a computing device 318 as in FIG. 3. Computing device 318 is one example of a computing device and is not intended to suggest any limitation as to scope of use or functionality of the computing device and/or its possible architectures. Neither should computing device 318 be interpreted as having any dependency or requirement relating to any one or combination of components illustrated in the example computing device 318.

Computing device 318 includes one or more processors or processing units 314, one or more memory/storage components 315, one or more input/output (I/O) devices 316, and a bus 317 that allows the various components and devices to communicate with one another. Bus 317 represents one or more of any of several types of bus structures, including a memory bus or memory controller, a peripheral bus, an accelerated graphics port, and a processor or local bus using any of a variety of bus architectures. Bus 317 includes wired and/or wireless buses.

Memory/storage component 315 represents one or more computer storage media. Memory/storage component 315 includes volatile media (such as random access memory (RAM)) and/or nonvolatile media (such as read only memory (ROM), flash memory, optical disks, magnetic disks, and so forth). Memory/storage component 315 includes fixed media (e.g., RAM, ROM, a fixed hard drive, etc.) as well as removable media (e.g., a Flash memory drive, a removable hard drive, an optical disk, and so forth).

One or more I/O devices 316 allow a customer, utility, or other user to enter commands and information to computing device 318, and also allow information to be presented to the customer, utility, or other user and/or other components or devices. Examples of input devices include, but are not limited to, a keyboard, a cursor control device (e.g., a mouse), a microphone, a touchscreen, and a scanner. Examples of output devices include, but are not limited to, a display device (e.g., a monitor or projector), speakers, outputs to a lighting network (e.g., DMX card), a printer, and a network card.

Various techniques are described herein in the general context of software or program modules. Generally, software includes routines, programs, objects, components, data structures, and so forth that perform particular tasks or implement particular abstract data types. An implementation of these modules and techniques are stored on or transmitted across some form of computer readable media. Computer readable media is any available non-transitory medium or non-transitory media that is accessible by a computing device. By way of example, and not limitation, computer readable media includes “computer storage media”.

“Computer storage media” and “computer readable medium” include volatile and non-volatile, removable and non-removable media implemented in any method or technology for storage of information such as computer readable instructions, data structures, program modules, or other data. Computer storage media include, but are not limited to, computer recordable media such as RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which is used to store the desired information and which is accessible by a computer.

The computer device 318 is connected to a network (not shown) (e.g., a LAN, a WAN such as the Internet, cloud, or any other similar type of network) via a network interface connection (not shown) according to some exemplary embodiments. Those skilled in the art will appreciate that many different types of computer systems exist (e.g., desktop computer, a laptop computer, a personal media device, a mobile device, such as a cell phone or personal digital assistant, or any other computing system capable of executing computer readable instructions), and the aforementioned input and output means take other forms, now known or later developed, in other exemplary embodiments. Generally speaking, the computer system 318 includes at least the minimal processing, input, and/or output means necessary to practice one or more embodiments.

Further, those skilled in the art will appreciate that one or more elements of the aforementioned computer device 318 is located at a remote location and connected to the other elements over a network in certain exemplary embodiments. Further, one or more embodiments is implemented on a distributed system having one or more nodes, where each portion of the implementation (e.g., control engine 206) is located on a different node within the distributed system. In one or more embodiments, the node corresponds to a computer system. Alternatively, the node corresponds to a processor with associated physical memory in some exemplary embodiments. The node alternatively corresponds to a processor with shared memory and/or resources in some exemplary embodiments.

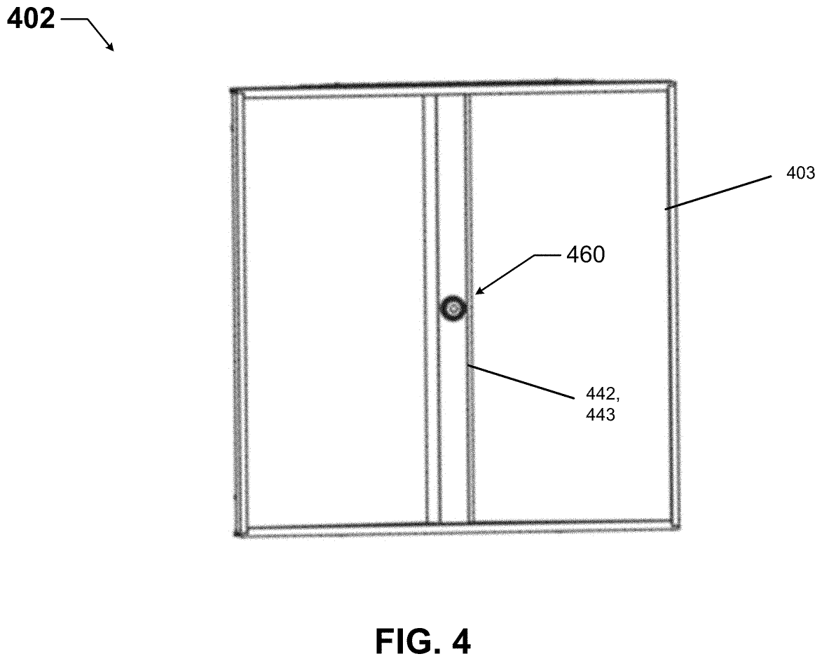

FIG. 4 shows a bottom view of an electrical device 402 (in this case, a light fixture) in accordance with certain example embodiments. Referring to FIGS. 1 through 4, the electrical device 402 of FIG. 4 includes a sensor module 460 that is coupled to the housing 403 of the light fixture 402. In this case, the sensor module 460 is an occupancy sensor that detects whether a person is in a volume of space (e.g., a room). The sensor module 460 protrudes outward from the housing 403 of the electrical device 402 (light fixture) and is visible when the electrical device 402 is installed.

The electrical device also includes both non-UV light sources 442 and UV light sources 443. When the sensor module 460 detects occupancy, the controller (not shown in FIG. 4, but substantially similar to the controller 204 of FIG. 2 above) of the electrical device 402 prevents the UV light sources 443 from operating. The controller may also control (e.g., turn on) the non-UV light sources 442 based on the occupancy (or some other parameter) detected by the sensor module 460. Alternatively, control of the non-UV light sources 442 by the controller may be independent of the occupancy (or some other parameter) measured by the sensor module 460.

Since the UV rays emitted by the UV light sources 443 are harmful to humans and other living beings, the controller of example electrical devices is configured to prevent the UV light sources 443 from operating when human and/or other living beings are determined to be in the vicinity of the electrical device 402. In some cases, when the UV light sources 443 are operating, there is no need for the non-UV light sources 442 to operate, and so the controller of the electrical device 402 can prevent the non-UV light sources 442 from operating when the UV light sources 443 are operating. Alternatively, as a way of indicating that the UV light sources 443 are emitting UV light, which is not visible to the human eye, the controller of the electrical device 402 can operate the non-UV light sources 442 in an atypical way (e.g., constant flashing, emit a red light) to warn a user that conditions are not safe in the volume of space.

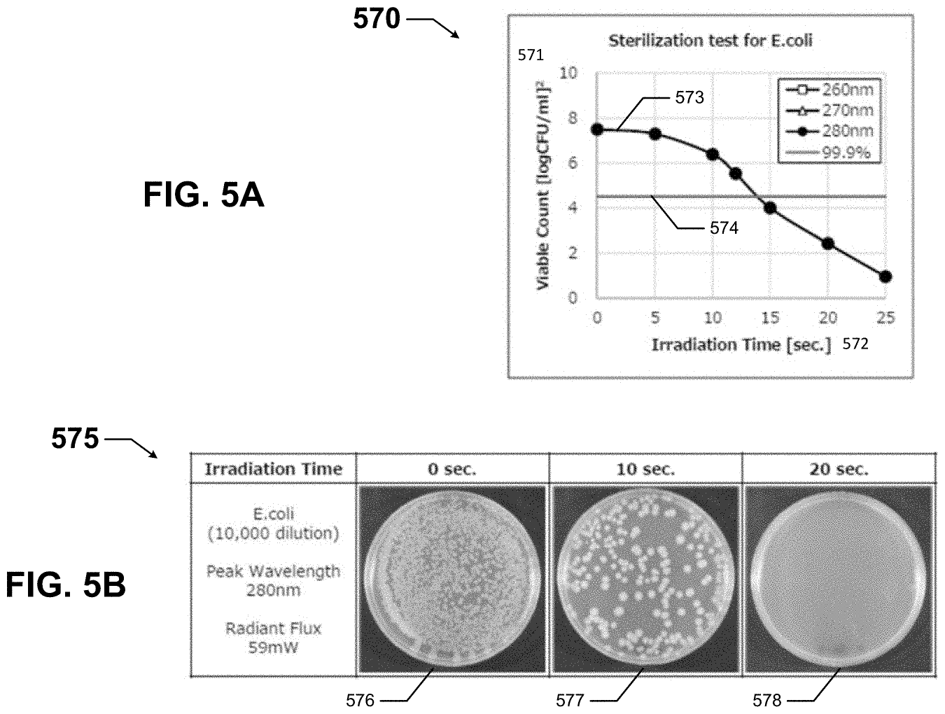

FIGS. 5A and 5B show a graph 570 and a table 575, respectively, as to the effectiveness of UV radiation exposure to E. coli bacteria using example embodiments. In other words, example embodiments can be used to kill E. coli bacteria within a volume of space. Referring to FIGS. 1 through 5B, the graph 570 of FIG. 5A shows a plot of irradiation time 572 (in seconds) along the horizontal axis and viable count 571 (in a logarithm of colony forming units per milliliter) of E. coli bacteria. Plot 573 shows the actual test data using UVC radiation having a wavelength of 280 nm, and plot 574 represents 99.9% irradiation, which is statistically seen as complete irradiation (or, as defined herein, disinfection). The graph 570 shows that complete irradiation of the E. coli bacteria occurs after approximately 14 seconds. The UVC radiation can be emitted by a UV light source (e.g., UV light source 243) of an example electrical device (e.g., electrical device 202), such as a light fixture.

The table 575 of FIG. 5B shows a pictorial image of the E. coli count at various points in time under the test conducted in FIG. 5A. Specifically, image 576 of FIG. 5B shows the E. coli count before the irradiation (the exposure of the E. coli bacteria to UVC light) has begun. Image 577 of FIG. 5B shows the E. coli count 10 seconds after the irradiation has begun. Image 578 of FIG. 5B shows the E. coli count 20 seconds after the irradiation has begun. The graph 570 and the table 575 show how effective UVC radiation is at eliminating harmful bacteria using example embodiments.

In certain example embodiments, a timer (e.g., timer 210) of a controller (e.g., controller 204) can keep track of the amount of time that UV light sources (e.g., UV light sources 243) of an electrical device (e.g., electrical device 202) have been operating. In such a case, by following some protocol (e.g., protocol 232), after a certain amount of time has elapsed, the volume of space (e.g., volume of space 219) subject to the UV light can be considered disinfected (e.g., according to an applicable standard or policy). When such time threshold has been reached, the controller of the electrical device can cause the UV light sources to stop operating, as further disinfection is not needed.

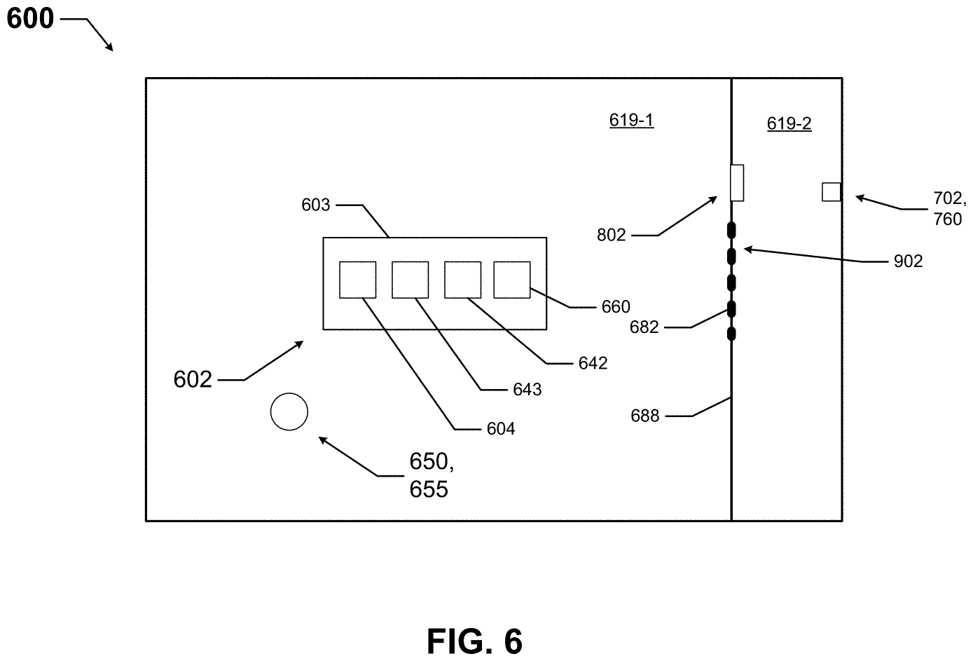

FIGS. 6 through 9 show examples in accordance with example embodiments. Referring to FIGS. 1 through 9, the systems shown in FIGS. 6 through 9 includes multiple components that are substantially the same as the corresponding components of the system 200 of FIG. 2 above. FIG. 6 shows a system 600 within a volume of space 619-1 in the form of a room and an adjacent volume of space 619-2 in the form of a hallway. Volume of space 619-1 and volume of space 619-2 are separated from each other by a wall 688 and a door 682 set in the wall 688.

In the system 600 of FIG. 6, there are three electrical devices in the volume of space 619-2, but they are not used in the example of FIG. 6. Specifically, disposed in the volume of space 619-2 are electrical device 702 in the form of, or that includes, a sensor module 760 that measures infrared radiation (for occupancy), electrical device 802 in the form of an access card reader or access keypad, and electrical device 902 in the form of an electrical lock for the door 682.

Within the volume of space 691-1 is a user 650 (along with a user system 655) and an electrical device 602. In this case, the electrical device 602 in this case is in the form of a ceiling-mounted light fixture. The electrical device 602 includes a housing 603, a controller 604, a sensor module 660 that measures infrared radiation (for occupancy), at least one non-UV light source 642, and at least one UV light source 643. The sensor module 660 detects the presence of the user 650 within the volume of space 619-1, and the controller 604 uses this information, following one or more protocols (e.g., protocols 232), to prevent the UV light sources 643 from operating. Whether the controller 604 also operates or prevents the operation of the non-UV light sources 642 can depend on one or more of a number of other factors, such as the amount of ambient light in the volume of space 619-1.

Even if there is harmful bacteria or other micro-organisms within the volume of space 619-1 that needs to be irradiated with UV light (e.g., UVA, UVB, UVC), the controller 604 prevents the UV light sources 643 from operating because the UV light is harmful to the user 650. If such a situation existed, the controller 604 can take actions to clear the user 650 from the volume of space 619-1 so that the UV light sources 643 can be safely operated to disinfect the volume of space 619-1. For example, the controller 604 could send a text message to the user system 655 of the user 650 when the user system 655 is a cell phone.

As another example, the controller 604 could emit an audible alarm or recorded speech through a speaker in the electrical device 602 or some other electrical device to instruct the user 650 to leave the volume of space 619-1. As yet another example, the controller 604 could cause the non-UV light sources 642 to strobe as an indication that the user 650 should leave the volume of space 619-1. As still another example, the controller 604 could contact security within the building, and subsequently a member of building security could go to the volume of space 619-1 to instruct the user 650 to vacate the volume of space 619-1.

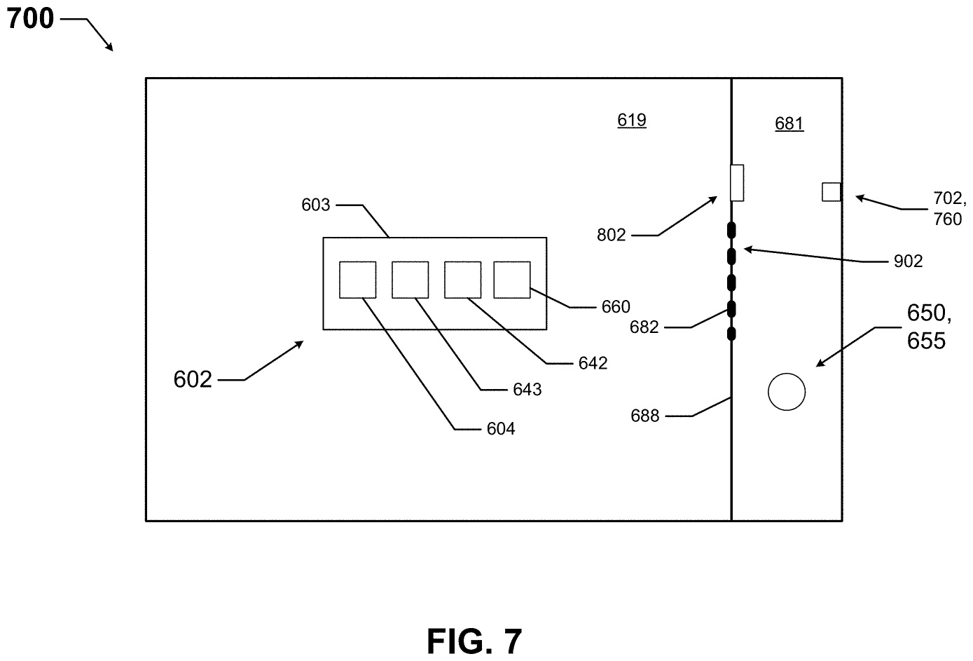

The system 700 of FIG. 7 is the same as the system 600 of FIG. 6, except that in this case, the user 650 (and associated user system 655) is located in the entry of the volume of space 619-2. As a result, there are no users within the volume of space 619-1, and so the controller 604 can cause the UV light sources 643 to operate in order to disinfect the volume of space 619-1. While the UV light sources 643 are operating, the controller 604 can perform one or more of a number of actions for safety of users, such as user 650. For example, while the UV light sources 643 are operating, the controller 604 can operate the electrical device 902 in the form of the electrical lock for the door 682, keeping the door 682 locked for as long as the UV light sources 643 emit UV light that is harmful to the user 650.

As another example, while the UV light sources 643 are operating, the controller 604 can communicate with electrical device 702 in such a way that, when the sensor module 760 of the electrical device 702 detects the presence of the user 650 in the volume of space 619-2, the controller 604 lock out the electrical device 802 and/or the electrical device 902 so that the user 650 is unable to enter the volume of space 619-1 until the UV light sources 643 are turned off. When the UV light sources 643 are no longer operating, the controller 604 can release its control of electrical device 802 and/or electrical device 902 so that they operate according to their normal protocols.

As yet another example, while the UV light sources 643 are operating, the controller 604 can automatically broadcast an announcement through a speaker in the electrical device 602, from the time that the UV light sources 643 until the time that the UV light sources 643 are turned off, that all users should avoid entering the volume of space 619-1. This announcement can be continuously repeated for the entirety of the time period. This announcement can also include, in real time, the amount of time remaining until the UV light sources 643 are turned off.

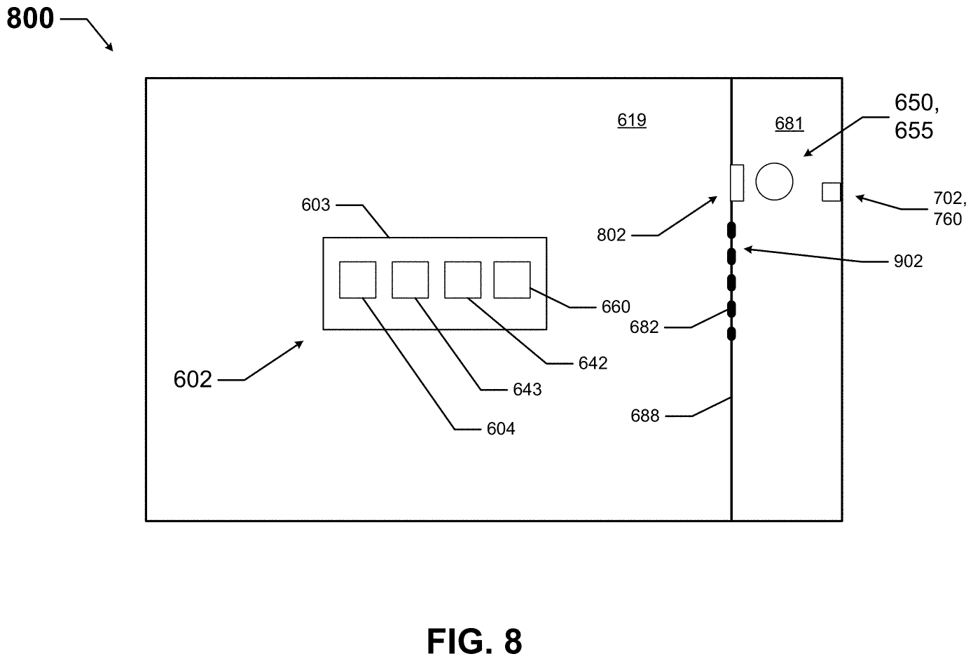

The system 800 of FIG. 8 is the same as the system 700 of FIG. 7, except that in this case, the user 650 (and associated user system 655) is located in the volume of space 619-2 in front of the electrical device 802. As a result, there are no users within the volume of space 619-1, and so the controller 604 can cause the UV light sources 643 to operate in order to disinfect the volume of space 619-1. While the UV light sources 643 are operating, the controller 604 can perform one or more of a number of actions for safety of users, such as user 650.

For example, while the UV light sources 643 are operating, the controller 604 can communicate with electrical device 802 and lock out any attempted access to the volume of space 619-1 through the door 682 by the user 650. For example, the controller 604 can determine that 3 minutes remain until the volume of space 619-1 is disinfected and instruct the electrical device 802 to broadcast a recording, when the user 650 attempts to enter an access code or show the user system 655 to a card reader, to state that access to the volume of space 619-1 will be denied for the next 3 minutes until the disinfection process is complete.

As another example, while the UV light sources 643 are operating, the controller 604 can communicate with a light fixture (e.g., electrical device 702) located in the volume of space 619-2 to emit light in a certain way (e.g., emit a non-white (e.g., red) color, emit light with a strobe effect) to signal to the user 650 that disinfection is occurring in the volume of space 619-1 and that entry into the volume of space 619-1 is not permitted at that time.

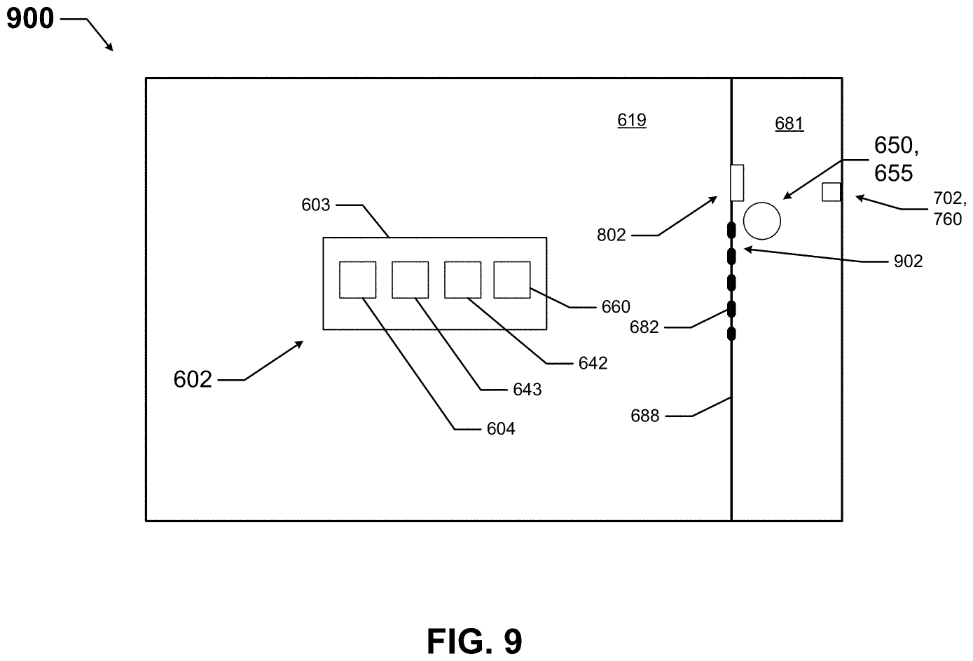

The system 900 of FIG. 9 is the same as the system 800 of FIG. 8, except that in this case, the user 650 (and associated user system 655) is located in the volume of space 619-2 in front of the door 682 and electrical device 902. As a result, there are no users within the volume of space 619-1, and so the controller 604 can cause the UV light sources 643 to operate in order to disinfect the volume of space 619-1. While the UV light sources 643 are operating, the controller 604 can perform one or more of a number of actions for safety of users, such as user 650.

For example, as mentioned above, while the UV light sources 643 are operating, the controller 604 can operate the electrical device 902 in the form of the electrical lock for the door 682, keeping the door 682 locked for as long as the UV light sources 643 emit UV light that is harmful to the user 650. In this way, even if the electrical device 802 allows the user 650 access to the volume of space 619-1, the controller 604 can deny that access by controlling the electrical device 902 until the UV light sources 643 are turned off.

In such a case, the controller 604 detect, in communications with electrical device 902, that the user 650 is attempting to open the door 682 using the door handle and subsequently communicate with the user 650 (e.g., by broadcasting an announcement over a nearby speaker, by sending a text message to the user system 655) why access to the volume of space 619-1 is being denied and when (e.g., in five minutes) access to the volume of space 619-1 will be granted.

As another example, if electrical device 902 is a sensor module that determines (e.g., using a proximity sensor) whether the door 682 is fully closed, the controller 604 can communicate with the electrical device 902 and only allow the UV light sources 643 to operate when the sensor module of electrical device 902 determines that the door 682 is fully closed. If at any time, including while the UV light sources 643 are emitting UV light into the volume of space 619-1, the sensor module of electrical device 902 determines that the door 682 is not fully closed, then the controller 604 can immediately cease operation of the UV light sources 643 (if they are in operation) or prevent the UV light sources 643 from becoming operational.

In one or more example embodiments, electrical devices have at least one UV light source that emits UV radiation at times when there is no human being (a type of user) in a volume of space in which the UV radiation is emitted. Example embodiments can be part of a newly manufactured electrical device (e.g., a light fixture), or alternatively example embodiments can be retrofitted in or to work with an existing electrical device. Example embodiments can use one or more sensor modules to determine whether the volume of space is occupied by a human or other living being, which determines whether it is safe to have the at least one UV light source to emit UV radiation into the volume of space. Example embodiments can provide safe and effective disinfection of various surfaces and objects in the volume of space. Using example embodiments described herein can improve safety, health, maintenance, costs, and operating efficiency.

Accordingly, many modifications and other embodiments set forth herein will come to mind to one skilled in the art to which example embodiments pertain having the benefit of the teachings presented in the foregoing descriptions and the associated drawings. Therefore, it is to be understood that example embodiments are not to be limited to the specific embodiments disclosed and that modifications and other embodiments are intended to be included within the scope of this application. Although specific terms are employed herein, they are used in a generic and descriptive sense only and not for purposes of limitation.

What is claimed is:

1. An electrical device comprising:
a sensor module that measures at least one parameter, wherein the at least one parameter is associated with determining the presence of a living being in a volume of space;
a controller coupled to the sensor module, wherein the controller receives a measurement of the at least one parameter to determine whether the living being is in the volume of space; and
at least one ultraviolet (UV) light source coupled to the controller, wherein the controller, upon determining that the living being is not in the volume of space, operates the at least one UV light source to emit UV light into the volume of space, and wherein the controller, upon determining that the living being is in the volume of space, prevents the at least one UV light source from emitting the UV light into the volume of space.

2. The electrical device of claim 1, wherein the at least one parameter comprises occupancy within the volume of space.

3. The electrical device of claim 1, wherein the at least one parameter comprises detecting that a door to the volume of space is opening.

4. The electrical device of claim 1, wherein the controller instructs the at least one UV light source to cease emitting the UV radiation after a period of time, measured by a timer, while the volume of space remains unoccupied.

5. The electrical device of claim 1, wherein the UV light emitted by the at least one UV light source is UVC radiation.

6. The electrical device of claim 1, further comprising:
at least one non-UV light source coupled to the controller, wherein the at least one non-UV light source emits non-UV light into the volume of space when operated by the controller.

7. The electrical device of claim 6, wherein the controller instructs the at least one UV light source to emit the UV light when the at least one non-UV light source is emitting a first non-UV light emission, wherein the first non-UV light emission comprises a color indicating that entry into the volume of space is unsafe.

8. The electrical device of claim 7, wherein the color is red.

9. The electrical device of claim 7, wherein the controller instructs the at least one non-UV light source to emit a second non-UV light emission when the at least one UV light source is turned off.

10. The electrical device of claim 6, wherein the controller instructs the at least one non-UV light source to emit a non-UV light emission when the at least one UV light source is turned off, and wherein the controller instructs the at least one UV light source to emit a UV light emission when the at least one non-UV light source is turned off.

11. The electrical device of claim 1, wherein the at least one UV light source is further configured to emit visible light radiation.

12. The electrical device of claim 11, wherein the controller directs the at least one UV light source to emit the visible light radiation when the volume of space is occupied by the living being, and wherein the controller directs the at least one UV light source to emit UV radiation when the volume of space is unoccupied by the living being.

13. The electrical device of claim 1, wherein the UV radiation disinfects objects located within the volume of space.

14. The electrical device of claim 1, wherein the living being is a human being.

15. A system comprising:
an electrical device disposed in a volume of space, wherein the electrical device comprises:
a sensor module that measures at least one parameter, wherein the at least one parameter is associated with determining the presence of a living being in a volume of space; and
a controller coupled to the sensor module and the at least one UV light source, wherein the controller receives a measurement of the at least one parameter to determine whether the living being is in the volume of space; and
at least one ultraviolet (UV) light source coupled to the controller, wherein the controller, upon determining that the living being is not in the volume of space, operates the at least one UV light source to emit UV light into the volume of space, and wherein the controller, upon determining that the living being is in the volume of space, instructs the at least one UV light source to stop emitting the UV light into the volume of space.

16. The system of claim 15, wherein the electrical device is a light fixture that provides general illumination to the volume of space when the living being is within the volume of space.

17. The system of claim 15, further comprising:
an additional electrical device in communication with the controller, wherein the controller controls the additional electrical device when the least one UV light source emits UV light into the volume of space.

18. The system of claim 17, wherein the additional electrical device comprises an electronic door lock.

19. The system of claim 17, wherein the additional electrical device comprises an access panel.

20. The system of claim 17, wherein the additional electrical device comprises an additional sensor module located in an adjacent volume of space.
